(19) United States
(12) Patent Application Publication
(10) Pub. No.: US 2023/0326567 A1
(43) Pub. Date: Oct. 12, 2023

(54) SYSTEM AND METHOD TO FACILITATE MEDICAL ADHERENCE

(71) Applicant: Adherelive Private Limited, Delhi (IN)

(72) Inventors: Vivek Asthana, Meerut (IN); Ankur Agrawal, Gurugram (IN)

(21) Appl. No.: 18/043,158

(22) PCT Filed: Aug. 27, 2021
(86) PCT No.: PCT/IB2021/057850

§ 371 Date: Feb. 27, 2023

(30) Foreign Application Priority Data
Aug. 28, 2020 (IN) 202041037115

(51) Int. Cl. G16H 20/00; G16H 50/30; G16H 40/67; G16H 15/00

(52) U.S. Cl. CPC G16H 20/00; G16H 50/30; G16H 40/67; G16H 15/00

(57) ABSTRACT

A system to facilitate medical adherence is disclosed. The system includes a patient information recording module, configured to record information regarding at least one of one or more diseases, one or more bio-physical parameters, type of discomfort, severity of discomfort, part of body experiencing the discomfort and time of discomfort associated with registered patient via at least one of textual input, pre-designated field input and input on 3D image representative of human body. The system includes a medical treatment module, configured to diagnose the one or more diseases, generate a real-time treatment plan and generate a digital prescription for the registered patient. The system includes a medical adherence module, configured to monitor adherence of the registered patient to the real-time generated treatment plan at a micro level, create a medical adherence report and trigger an alert on an event of non-adherence of the real-time generated treatment plan.

### EARLIEST PRIORITY DATE

This application claims priority from a complete patent application filed in India having Patent Application No. 202041037115, filed on Aug. 28, 2020, and titled “SYSTEM AND METHOD TO FACILITATE MEDICAL ADHERENCE” and claims priority from a PCT patent application having Patent Application No. PCT/IB2021/057850, filed on Aug. 27, 2021, and titled “SYSTEM AND METHOD TO FACILITATE MEDICAL ADHERENCE”.

### FIELD OF INVENTION

Embodiments of a present disclosure relates to a field of healthcare, and more particularly to a system and a method to facilitate medical adherence.

### BACKGROUND

One of the most vital development in healthcare industry is the recent integration of information technology with the healthcare system, whereby the operation or interaction has become smooth between doctors and patients. Major advantage of such integration is digitization of medical records or any associated medical data.

It is important to note that such integration in no way helps in enforcing patient compliance with prescription, health care regimens and therapies. Conventional known healthcare systems only enable interaction facility between the doctors and patients, but no adherence. Such conventional systems enable healthcare workers, hospitals or clinics to preserve proper digitized records of any patient's medical history for future consideration. However, the process of ensuring a patient to adhere to a treatment plan including prescriptions, tests and regular check-ups etc. is manual. The adherence of the patients to treatment plans is very critical for speedy recuperation of the patient and assessment of medical effectiveness of a given treatment plan for a particular disease and age group. Such information plays critical role in improving treatment plans and prescribing of right dosages of medicines. Therefore, for creating a reliable medical infrastructure, a real time adherence tracking system is of utmost importance.

Hence, there is a need for an improved system to facilitate medical adherence and a method to operate the same and therefore address the aforementioned issues.

### BRIEF DESCRIPTION

In accordance with one embodiment of the disclosure, a system to facilitate medical adherence is disclosed. The system includes an entity registration module operable by the one or more processors. The entity registration module is configured to register one or more entities with a plurality of information. The system also includes a patient information recording module operable by the one or more processors. The patient information recording module is operatively coupled to the entry registration module. The patient information recording module is configured to record information regarding at least one of one or more diseases, one or more bio-physical parameters, type of discomfort, severity of discomfort, part of body experiencing the discomfort and time of discomfort associated with registered patient via at least one of textual input, pre-designated field input and input on 3D image representative of human body.

The patient information recording module is also configured to record real-time information regarding the one or more diseases and the one or more bio-physical parameters of the registered patient via IOT enabled one or more wearable sensors. The system also includes a medical treatment module operable by the one or more processors. The medical treatment module is operatively coupled to the patient information recording module. The medical treatment module is configured to diagnose the one or more diseases based on one or more recorded bio-physical parameters, type of recorded discomfort, severity of recorded discomfort, part of body experiencing the recorded discomfort and time of recorded discomfort associated with the registered patient.

The medical treatment module is also configured to generate a real-time treatment plan for the registered patient corresponding to each of one or more diagnosed diseases, based on a pre-designed one or more treatment templates, upon receiving approval from at least one of registered healthcare personnel and the registered doctor. The medical treatment module is also configured to generate a digital prescription for the registered patient according each of the one or more diagnosed diseases and corresponding real-time generated treatment plan.

The system also includes a medical adherence module operable by the one or more processors. The medical adherence module is operatively coupled to the medical treatment module. The medical adherence module is configured to monitor adherence of the registered patient to the real-time generated treatment plan at a micro level and create a medical adherence report. The medical adherence module is also configured to trigger an alert on an event of non-adherence of the real-time generated treatment plan. The medical adherence module is also configured to escalate the alert by changing alert level priority on the event of non-compliance to a triggered alert.

The system also includes an emergency notification module operable by the one or more processors. The emergency notification module is operatively coupled to the medical adherence module. The emergency notification module is configured to notify at least one of the registered patients, the registered healthcare personnel, the registered doctor, registered administrator and registered patient contact about the registered patient adherence to the real-time generated treatment plan.

The system also includes a storage module operable by the one or more processors. The storage module is operatively coupled to emergency notification module. The storage module is configured to store one or more recorded diseases, one or more recorded bio-physical parameters, type of recorded discomfort, severity of recorded discomfort, part of body experiencing the recorded discomfort, time of recorded discomfort associated with registered patient, the real-time generated treatment plan corresponding to the recorded disease, the generated digital prescription corresponding to the recorded disease and created medical adherence report corresponding to the recorded disease.

The system also includes a treatment template generation module operable by the one or more processors. The treatment template generation module is operatively coupled to the storage module. The treatment template generation module is configured to design one or more treatment templates for a particular disease based on one or more stored diseases, one or more stored bio-physical parameters, type of stored discomfort, severity of stored discomfort, part of body experiencing the stored discomfort, time of stored discomfort associated with registered patient, the real-time generated treatment plan corresponding to the stored disease, generated digital prescription corresponding to the stored disease and created medical adherence report corresponding to the stored disease.

In accordance with one embodiment of the disclosure, a method for facilitating medical adherence is disclosed. The method also includes registering one or more entities upon receiving a plurality of information. The method also includes recording information regarding at least one of one or more diseases, one or more bio-physical parameters, type of discomfort, severity of discomfort, part of body experiencing the discomfort and time of discomfort associated with registered patient via at least one of textual input, pre-designated field input and 3-d image input representative of human body.

The method also recording real-time information regarding the one or more diseases and the one or more bio-physical parameters of the registered patient via IOT enabled one or more wearable sensors. The method also includes diagnosing the one or more diseases based on one or more recorded bio-physical parameters, type of recorded discomfort, severity of recorded discomfort, part of body experiencing the recorded discomfort and time of recorded discomfort associated with the registered patient. The method also generating a real-time treatment plan for the registered patient corresponding to each of one or more diagnosed diseases, based on a pre-designed one or more treatment template, upon receiving approval from at least one of registered healthcare personnel and the registered doctor.

The method also includes generating a digital prescription for the registered patient according each of the one or more diagnosed diseases and corresponding real-time generated treatment plan. The method also includes monitoring adherence of the registered patient to the real-time generated treatment plan at a micro level. The method also includes creating a medical adherence report. The method also includes triggering an alert on the event of non-adherence of the real-time generated treatment plan.

The method also includes escalating the alert by changing alert level priority on the event of non-compliance to triggered alert. The method also includes notifying at least one of the registered patients, the registered healthcare personnel, the registered doctor, registered administrator and registered patient contact about the registered patient adherence to the real-time generated treatment plan.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a block diagram representation of a system to facilitate medical adherence in accordance with an embodiment of the present disclosure;

FIG. 2 is a schematic representation of an embodiment representing input mechanism related to disease corresponding to a registered patient of FIG. 1 in accordance with an embodiment of the present disclosure;

FIG. 3 is a schematic representation of an embodiment representing a medical treatment plan corresponding to the registered patient of FIG. 1 in accordance with an embodiment of the present disclosure;

FIG. 4 is a schematic representation of an embodiment representing a digital prescription corresponding to the registered patient of FIG. 1 in accordance with an embodiment of the present disclosure;

FIG. 5 is a schematic representation of an embodiment representing an adherence dashboard window of FIG. 1 in accordance with an embodiment of the present disclosure;

FIG. 6 is a schematic representation of an embodiment representing the adherence dashboard window corresponding to the registered patient of FIG. 1 in accordance with an embodiment of the present disclosure;

FIG. 7 is a schematic representation of an embodiment representing the adherence dashboard window with real time information corresponding to the registered patient of FIG. 1 in accordance with an embodiment of the present disclosure;

FIG. 8 is a schematic representation of an embodiment representing a notification generation process corresponding to the registered patient having COVID 19 symptoms of FIG. 1 in accordance with an embodiment of the present disclosure;

FIG. 9 is a schematic representation of an embodiment representing a notification window corresponding to the registered patient of FIG. 1 in accordance with an embodiment of the present disclosure;

FIG. 10 is a schematic representation of a system to facilitate medical adherence of FIG. 1 in accordance with an embodiment of the present disclosure;

FIG. 11 is a block diagram of a computer or a server in accordance with an embodiment of the present disclosure;

FIG. 12 is a flowchart representing the steps of a method to facilitate medical adherence in accordance with an embodiment of the present disclosure; and

FIG. 13 is a flowchart representing the steps of the method to facilitate medical adherence in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated online platform, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or subsystems or elements or structures or components preceded by “comprises . . . a” does not, without more constraints, preclude the existence of other devices, subsystems, elements, structures, components, additional devices, additional subsystems, additional elements, additional structures or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

Embodiments of the present disclosure relate to a system to facilitate medical adherence. The system includes various modules to perform a specific set of functions such as registering various entities such as patients, doctors, and caregivers etc., recording information regarding one or more diseases, one or more bio-physical parameters, type of discomfort, severity of discomfort, part of body experiencing the discomfort and time of discomfort associated with registered patient via at least one of textual input, pre-designated field input and input on 3D image representative of human body. The system may record real-time information regarding the one or more diseases and the one or more bio-physical parameters of the registered patient via IoT enabled one or more wearable sensors. The system also diagnoses the one or more diseases based on one or more recorded bio-physical parameters, type of recorded discomfort, severity of recorded discomfort, part of body experiencing the recorded discomfort and time of recorded discomfort associated with the registered patient.

The system generates a real-time treatment plan and digital prescriptions for the registered patient corresponding to each of one or more diagnosed diseases, based on a pre-designed one or more treatment templates, upon receiving approval from at least one of registered healthcare personnel and the registered doctor.

The system monitors adherence of the registered patient to the real-time generated treatment plan at a micro level and create a medical adherence report. The system may trigger an alert on an event of non-adherence of the real-time generated treatment plan and escalate the alert by changing alert level priority on the event of non-compliance to a triggered alert. The system notifies the registered patients, the registered healthcare personnel, the registered doctor, registered administrator and registered patient contact about the registered patient adherence to the real-time generated treatment plan.

The system stores one or more recorded diseases, one or more recorded bio-physical parameters, type of recorded discomfort, severity of recorded discomfort, part of body experiencing the recorded discomfort, time of recorded discomfort associated with registered patient, the real-time generated treatment plan corresponding to the recorded disease, the generated digital prescription corresponding to the recorded disease and created medical adherence report corresponding to the recorded disease.

Further, the system also designs one or more treatment templates for a particular disease based on one or more stored diseases, one or more stored bio-physical parameters, type of stored discomfort, severity of stored discomfort, part of body experiencing the stored discomfort, time of stored discomfort associated with registered patient, the real-time generated treatment plan corresponding to the stored disease, generated digital prescription corresponding to the stored disease and created medical adherence report corresponding to the stored disease.

A computer system (standalone, client or server computer system) configured by an application may constitute a “platform” or “module’ that is configured and operated to perform certain operations. In one embodiment, the “platform” or “module’ may be implemented mechanically or electronically, so a platform or module may comprise dedicated circuitry or logic that is permanently configured (within a special-purpose processor) to perform certain operations. In another embodiment, a “platform” or “module” may also comprise programmable logic or circuitry (as encompassed within a general-purpose processor or other programmable processor) that is temporarily configured by software to perform certain operations.

Accordingly, the term “module’ or “platform’ should be understood to encompass a tangible entity, be that an entity that is physically constructed permanently configured (hardwired) or temporarily configured (programmed) to operate in a certain manner and/or to perform certain operations described herein.

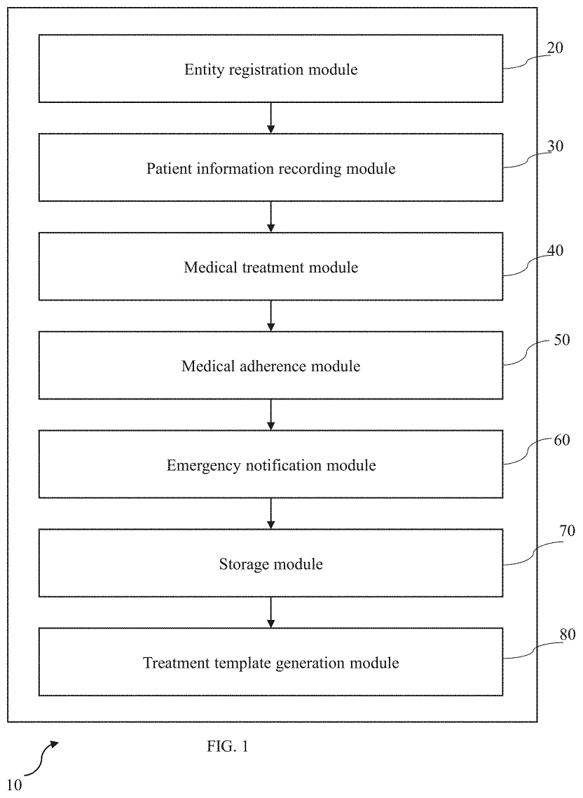

FIG. 1 is a block diagram representation of a system 10 to facilitate medical adherence in accordance with an embodiment of the present disclosure. Medical adherence refers to the degree to which a patient correctly follows the medical advice. In such embodiment, the medical adherence is vital for any patient for recovery from ailment. Adherence to a medication regimen and particularly to a prescribed medication regimen is an essential and critically important element to regimen efficacy and a desired therapeutic outcome.

The system 10 includes an entity registration module 20 operable by the one or more processors. The entity registration module 20 is configured to register one or more entities with a plurality of information. In one embodiment, the one or more entities comprises a patient, a healthcare personnel, a doctor, an administrator and a patient contact. In such embodiment, each of the one or more entities will have to provide specific information for registration.

In one specific embodiment, the patient for registration may provide information such as name, age, contact address, present location, past medical history and the like. In another specific embodiment, the doctor for registration may provide information such as name, contact address, area of specialization and the like. In yet another specific embodiment, the healthcare personnel for registration may provide information such as name, contact details, area of specialization, location and the like. The one or more healthcare personnel may include allied healthcare personnel, geriatric care personnel and the like. As used herein, the term “administrator” refers to a person responsible for carrying out the administration of the adherence system 10. Here, patient contact refers to a person responsible with health liability of registered patient.

The system 10 also includes a patient information recording module 30 operable by the one or more processors. The patient information recording module 30 is operatively coupled to the entry registration module 20. The patient information recording module 30 is configured to record information regarding at least one of one or more diseases, one or more bio-physical parameters, type of discomfort, severity of discomfort, part of body experiencing the discomfort and time of discomfort associated with registered patient via at least one of textual input, pre-designated field input and input on 3D image representative of human body.

As used herein, the term “disease” refers to a disorder of structure or function in a human, especially one that produces specific symptoms or that affects a specific location and is not simply a direct result of physical injury. The system 10 via the patient information recording module 30 records any specific disease affecting the registered patient body along with additional medical details. Discomfort may be the ongoing pain or uneasiness caused by the recorded disease. Detailed information regarding discomfort is also recorded via the patient information recording module 30.

In one specific embodiment, the registered patient may provide all medical information as stated via textual input. In such embodiment, the patient information recording module 30 uses data extraction techniques to look deeper into provided textual inputs to classify and extract relevant information in a structured manner. After data extraction, the input information is interpreted for understanding the disease or discomfort related to the recorded disease.

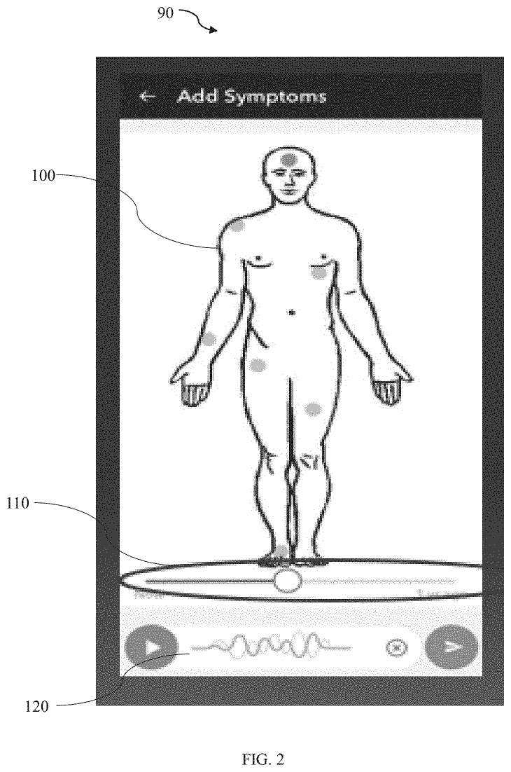

In another specific embodiment, an easy approach would be to provide an input via 3D image representative of human body. FIG. 2 is a schematic representation of an embodiment representing input mechanism window 90 related to disease corresponding to a registered patient of FIG. 1 in accordance with an embodiment of the present disclosure.

In such specific embodiment, the registered patient may provide information to the adherence system 10 via text input through a text or voice input bar 120. The text or voice input bar 120 is located at the bottom end of the recording input window 90. It is pertinent to note that, the registered patient may also provide voice input as well as pictorial input via the text or voice input bar 120. In such specific embodiment, all such details provided by the registered patient may be showcased on the 3d image body 100 as shown in FIG. 2. In another specific embodiment, the 3d image body may also be rotated or maximized for detail input via a slider 110. The registered doctor may easily use such features to get more detailed understanding of the recorded disease with respect to the registered patient.

The patient information recording module 30 is also configured to record real-time information regarding the one or more diseases and the one or more bio-physical parameters of the registered patient via IoT enabled one or more wearable sensors. In one embodiment, the IoT enabled one or more wearable sensors include smart watch, smart bangle and the like. In such embodiment, any bio-physical parameter such as heart rate, body pressure level may be easily be recorded via the patient information recording module 30 in real time for better tracking. IOT enabled one or more wearable sensors straightaway provide all recorded information to the respective registered patient adherence system 10.

The system 10 also includes a medical treatment module 40 operable by the one or more processors. The medical treatment module 40 is operatively coupled to the patient information recording module 30. The medical treatment module 40 is configured to diagnose the one or more diseases based on one or more recorded bio-physical parameters, type of recorded discomfort, severity of recorded discomfort, part of body experiencing the recorded discomfort and time of recorded discomfort associated with the registered patient. It is pertinent to not that the registered doctor in the system, identify the nature of (an illness or other problem) by examining the recorded symptoms.

The medical treatment module 40 is also configured to generate a real-time treatment plan for the registered patient corresponding to each of one or more diagnosed diseases, based on a pre-designed one or more treatment templates, upon receiving approval from at least one of registered healthcare personnel and the registered doctor. Here, the treatment method is presented o generated after proper authorization from the at least one of registered healthcare personnel and the registered doctor.

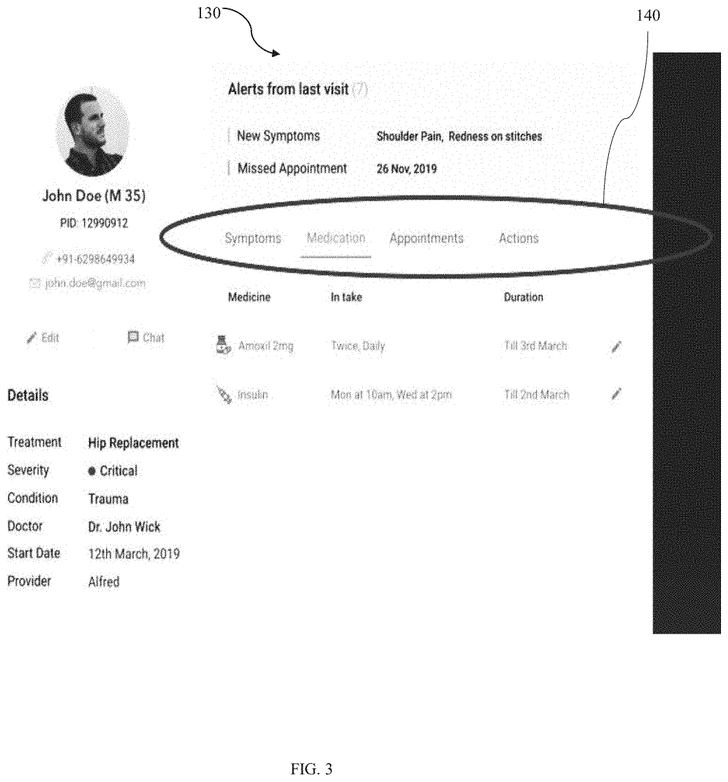

FIG. 3 is a schematic representation of an embodiment representing a medical treatment plan 130 corresponding to the registered patient of FIG. 1 in accordance with an embodiment of the present disclosure. In one embodiment, one or more recorded bio-physical parameters, type of recorded discomfort, severity of recorded discomfort, part of body experiencing the recorded discomfort and time of recorded discomfort may together be represented in one window for proper diagnose of a disease 140. The medical treatment plan additional provides details of last appointment, doctors name, contact details of the patient and the like. Past medical plan of the registered patient may also be taken into consideration for generating the medical treatment plan. Here, the pre-designed one or more treatment templates are used from the earlier stored treatment plan for the same disease.

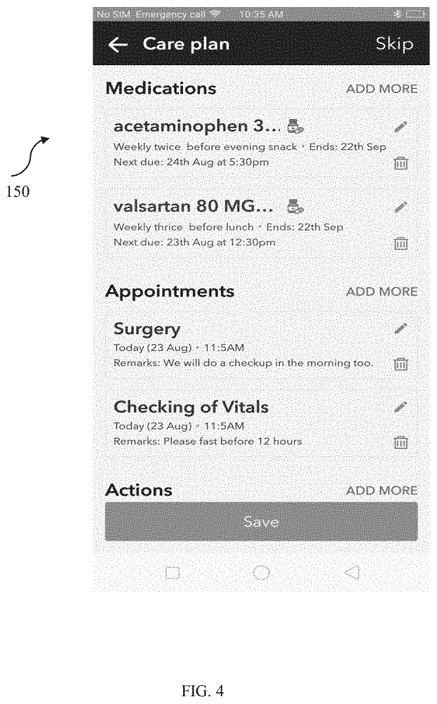

The medical treatment module 40 is also configured to generate a digital prescription for the registered patient according each of the one or more diagnosed diseases and corresponding real-time generated treatment plan. FIG. 4 is a schematic representation of an embodiment representing a digital prescription 150 corresponding to the registered patient of FIG. 1 in accordance with an embodiment of the present disclosure. It is pertinent to note that generated digital prescription 150 are digitally signed by the registered doctor for proper authorization.

The digital prescription will include details like dosage information, start date of medicine, end date of medicine, appointment details and the like. In one embodiment, the digital prescription may also provide information about the nearest available pharmacy shop with supply of the prescribed medicine utilizing the registered patient location. In such embodiment, the prescribing doctors may add the pharmacy details.

The system 10 also includes a medical adherence module 50 operable by the one or more processors. The medical adherence module 50 is operatively coupled to the medical treatment module 40. The medical adherence module 50 is configured to monitor adherence of the registered patient to the real-time generated treatment plan at a micro level. The medical adherence module 50 is configured to create a medical adherence report. In one embodiment, wherein the adherence comprises monitoring daily medication intakes, monitoring follow-up treatment compliance, monitoring medical appointments and monitoring ongoing symptoms and side-effects and the like.

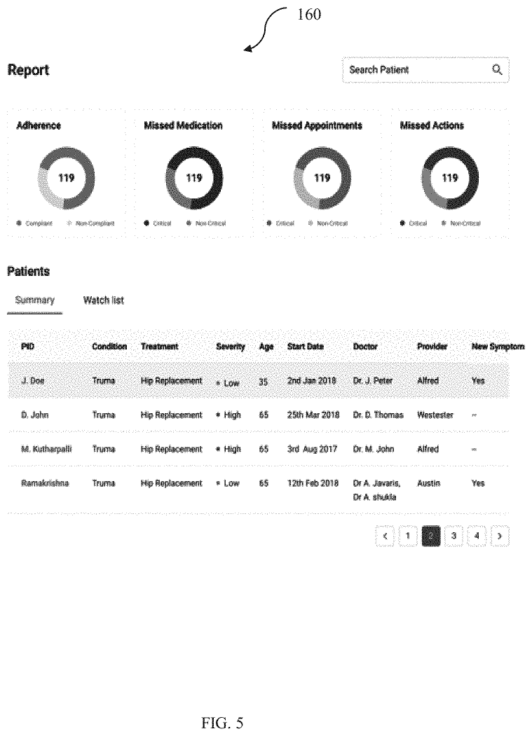

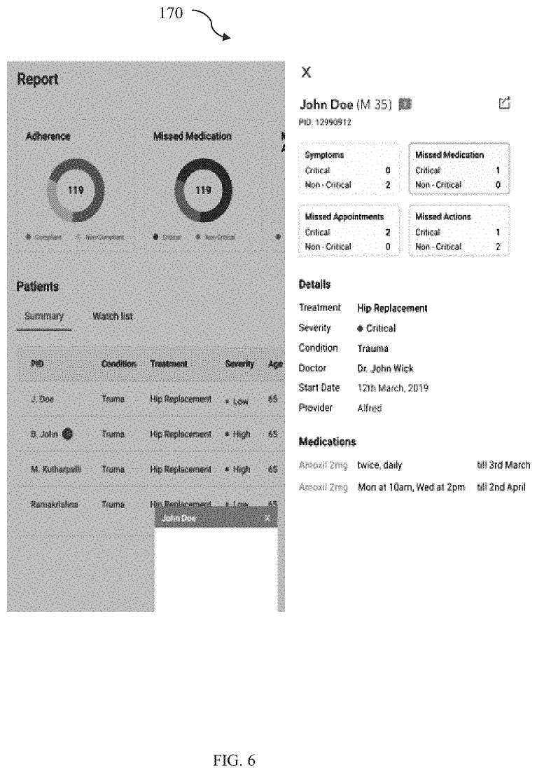

FIG. 5 is a schematic representation of an embodiment representing an adherence dashboard window 160 of FIG. 1 in accordance with an embodiment of the present disclosure. FIG. 6 is a schematic representation of an embodiment representing the adherence dashboard window 170 corresponding to the registered patient of FIG. 1 in accordance with an embodiment of the present disclosure. The adherence dashboard window 160 and 170 basically provides all the information related to adherence in a presentable format.

The adherence dashboard window 160 and 170 provides details like missed appointment, missed actions, missed medication and the like. In such embodiment, the system 10 in real time via notification gathers information about adherence of real time treatment plan. For example, the system 10 might generate yes or no notification for intake of medicine at particular time of the day. Here, on reply of the notification from the registered patient, the system 10 might update the adherence dashboard. Also, the adherence report might be created daily or monthly to understand healthcare advancement of the registered patient.

The medical adherence module 50 is also configured to trigger an alert on an event of non-adherence of the real-time generated treatment plan. The medical adherence module 50 is also configured to escalate the alert by changing alert level priority on the event of non-compliance to a triggered alert.

In one embodiment, the trigger of the alert on the event of non-adherence of the real-time generated treatment plan is notified to the registered patient. In such embodiment, on the event of non-compliance to the triggered alert the medical adherence module 50 will escalate a first priority alert to the registered healthcare personnel. Furthermore, on the event of non-compliance to the first priority alert the medical adherence module 50 will further escalate a second priority alert to the registered doctor. Here, healthcare personnel include nurses, dieticians and the like.

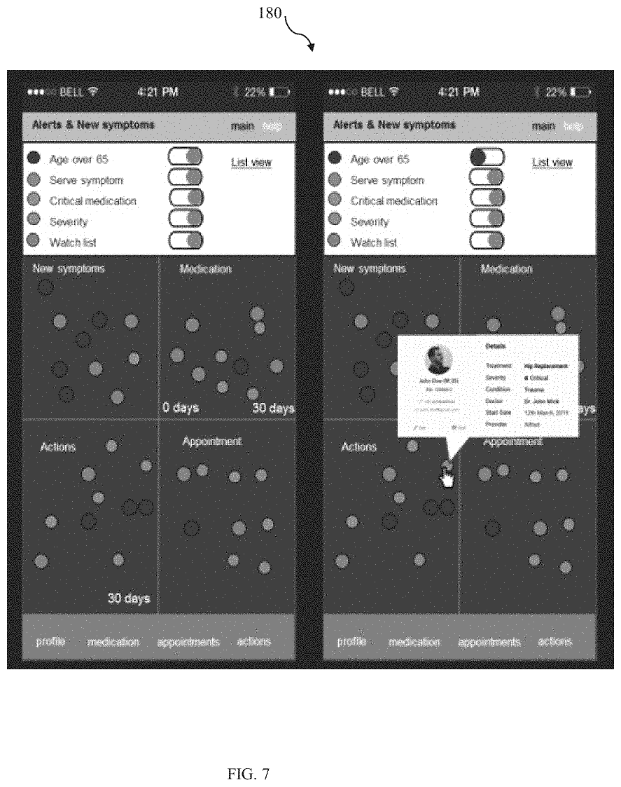

FIG. 7 is a schematic representation of an embodiment representing the adherence dashboard window 180 with real time information corresponding to the registered patient of FIG. 1 in accordance with an embodiment of the present disclosure. The adherence dashboard window 180 basically divides the presentation unit into multiple parts and provides details regarding medication, appointments and symptoms for multiple registered patients. Here, the registered doctor may be able to view information regarding all patients under treatment and registered in the system. Furthermore, the registered doctor may also be able to filter above stated information via one or more criteria. The one or more criteria as stated may include severity, age, location, watchlist and the like.

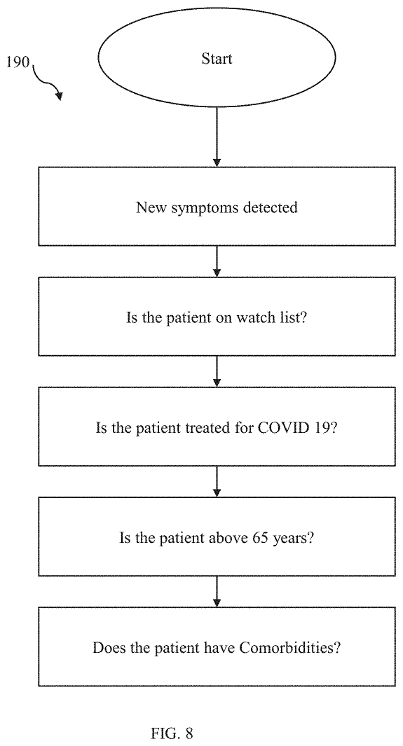

FIG. 8 is a schematic representation of an embodiment representing the notification generation process 190 corresponding to the registered patient having COVID 19 symptoms in accordance with an embodiment of the present disclosure. For example, to understand or create a proper real time treatment plan, the adherence system 10 may have to analyse many real time questions regarding the diseases.

For specific COVID 19 disease, the system 10 may have to know from the registered patient information such as “Is the patient on watchlist or not?”, “Is the patient above 65 years aga or not ?” and “Is the patient have any comorbidity or not ?”. Such information is vital for developing proper treatment plan or prescription in real time. All such real time information may be gathered via the system notification process only. In some embodiment, the system 10 may use pre-recorded information of the registered patient in question.

The system 10 also includes an emergency notification module 60 operable by the one or more processors. The emergency notification module 60 is operatively coupled to the medical adherence module 50. The emergency notification module is configured to notify at least one of the registered patient, the registered healthcare personnel, the registered doctor, registered administrator and registered patient contact about the registered patient adherence to the real-time generated treatment plan.

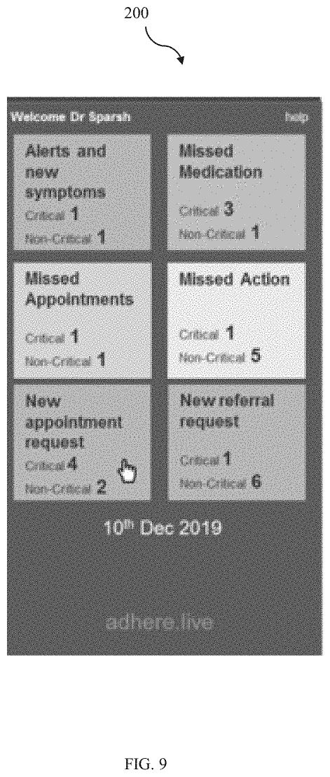

FIG. 9 is a schematic representation of an embodiment representing a notification window 200 corresponding to the registered patient of FIG. 1 in accordance with an embodiment of the present disclosure. In such embodiment, the normal notification may be presented to each of the one or more entities via a pop-up message, electronic mail and the like. In one embodiment, the normal notification and emergency notifications may be the same.

The system 10 also includes a storage module 70 operable by the one or more processors. The storage module 70 is operatively coupled to emergency notification module 60. The storage module 70 is configured to store one or more recorded diseases, one or more recorded bio-physical parameters, type of recorded discomfort, severity of recorded discomfort, part of body experiencing the recorded discomfort, time of recorded discomfort associated with registered patient, the real-time generated treatment plan corresponding to the recorded disease, the generated digital prescription corresponding to the recorded disease and created medical adherence report corresponding to the recorded disease. Here, the storage may be remote storage or local storage.

The system 10 also includes a treatment template generation module 80 operable by the one or more processors. The treatment template generation module 80 is operatively coupled to the storage module 70. The treatment template generation module 80 is configured to design one or more treatment templates for a particular disease based on one or more stored diseases, one or more stored bio-physical parameters, type of stored discomfort, severity of stored discomfort, part of body experiencing the stored discomfort, time of stored discomfort associated with registered patient, the real-time generated treatment plan corresponding to the stored disease, generated digital prescription corresponding to the stored disease and created medical adherence report corresponding to the stored disease. It is pertinent to note that such designed templates may be used by the medical treatment module for generating treatment plan. For designing template, the system 10 may at first analyse the stored details and accordingly design templates of various diseases. For analysing the templates, the system 10 may use data extraction technique and machine learning technique.

The system 10 also includes an entity interaction module operable by the one or more processors. The entity interaction module is operatively coupled to the medical adherence module 50. The entity interaction module is configured to provide textual or call interaction facility among the one or more registered entities.

The system 10 also includes a feedback module operable by the one or more processors. The feedback module is operatively coupled to the medical adherence module 50. The feedback module is configured to facilitate exchange of real-time feedbacks among the one or more registered entities, wherein the real-time feedbacks enable constant monitoring the registered patient health condition. Such real time feedbacks are vital for monitoring the registered patient health.

The system 10 also includes a health report generation module operable by the one or more processors. The health report generation module is operatively coupled to the emergency notification module 60. The health report generation module is configured to generate a health report based on the real-time generated treatment plan, the generated digital prescription and the created adherence report.

The system 10 also includes a research repository and sharing module operable by the one or more processors. The research repository and sharing module is operatively coupled to the medical adherence module 50. The research repository and sharing module is configured to store medical data corresponding to one or more diseases from a plurality of patients in at least one of a canonical and non-canonical data models. For storing the data, the research repository and sharing module may directly interact with the storage module 70.

The research repository and sharing module is also configured to categorise the stored medical data as per privacy levels. The research repository and sharing module is configured to provide access to the stored medical data, based on a clearance with respect to predefined privacy levels, to one or more entities. Here, the privacy levels being defined with respect to privacy concerns and research requirements. In one exemplary embodiment, the registered patient may deny permission to share medical information or to other patients or doctors or medical research organizations in-accordance to the need. for social concerns.

The system 10 further includes a triage module configured to categorise the plurality of parties based on degrees of urgency. Whereby the parties, according to pre-established category may access the medical information of the registered patient. In one exemplary embodiment, the registered doctor may have access to certain important medical information that are vital for diagnosing any particular disease. In such exemplary embodiment, the registered healthcare personnel may not have access to the stated file.

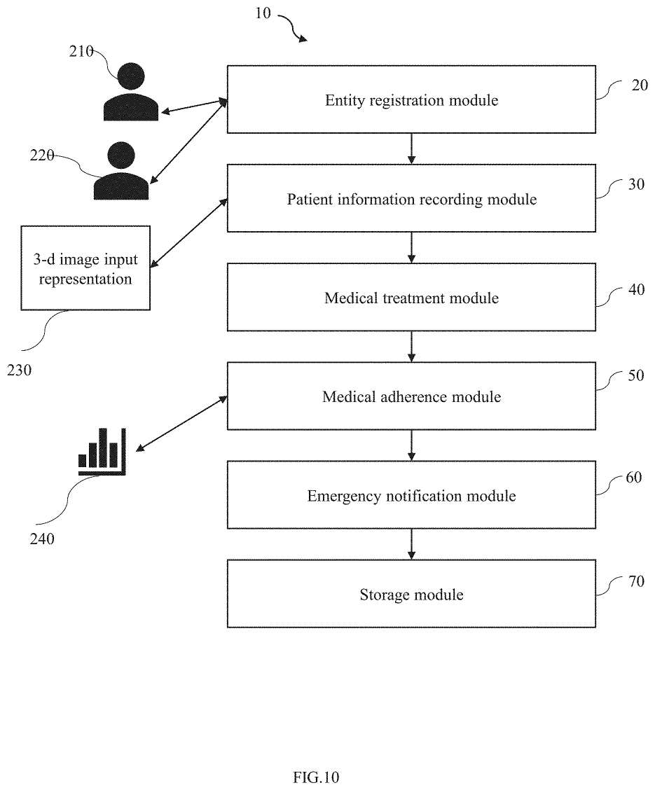

FIG. 10 is a schematic representation of the system 10 to facilitate medical adherence of FIG. 1 in accordance with an embodiment of the present disclosure. In one exemplary embodiment, a patient X 210 registers in the adherence system 10 for a doctor or a healthcare personnel 220 consultation. During registration, the patient X 210 via an entity registration module 20 provides name, age, contact details, comorbidity details and the like. Meanwhile, the doctor or the healthcare personnel 220 also register via the entity registration module 20 or may be pre-registered.

The registered patient X 210 through a patient information recording module 210 provides symptoms of a disease to the adherence system 10. The patient X 210 provides information such as abdominal pain that radiates to back, loss of appetite or unintended weight loss, yellowing of your skin and the whites of your eyes (jaundice), light-coloured stools, dark-coloured urine, itchy skin, and fatigue. The registered patient X 210 may input all details via a 3d image input representation 230.

The registered doctor or the registered healthcare personnel 220 may start the treatment plan by suggesting tests such as pancreas biopsy test and many more. It is pertinent to note that the registered doctor and the healthcare personnel 220 first diagnose the problem of the registered patient X 210 body and then prescribes the prescription. After the test are done, the registered doctor or the registered healthcare personnel 220 may provide digital prescription based on the results.

In such example, if the pancreas biopsy test shows pancreatic cancer, the registered doctor and the healthcare personnel 220 may suggest surgery or medication treatment plan in accordance with cancer stage. In both cases, via a medical adherence module, the system monitors medical adherence of the registered patient X 210.

For medication treatment plan, the system 10 monitors in real time the medication intake of the registered patient X 210. Whereby the real time questions through notification is asked to the registered patient X 210. Such notification enables the system 10 to understand where the medication is in time or not, any more side effects, and additional treatment is required or not and the like.

Similarly, if surgery path is chosen, the system 10 after surgery will monitor post-surgery medication treatment plan. It is important to note that automatic comprehension is very much required for adherence, as for any non-adherence the system 10 via the emergency notification module 60 may trigger alert.

The alert triggered may be set in different level priority on the basis of response, so that the registered patient X 210 health is not ignored in any step. The system 10 also creates the daily adherence report to understand the improvement of the registered patient X 210 health. Here, generated medication treatment plan, generated prescription and the adherence repot is stored by a storage module 70 in the system for later usage. The storage module 70 additionally stores symptoms and health problem details of the registered patient X 210.

The entity registration module 20, the patient information recording module 30, the medical treatment module 40, the medical adherence module 50, the emergency notification module 60 and the storage module 70 in FIG. 10 is substantially equivalent to the entity registration module 20, the patient information recording module 30, the medical treatment module 40, the medical adherence module 50, the emergency notification module 60 and the storage module 70 of FIG. 1.

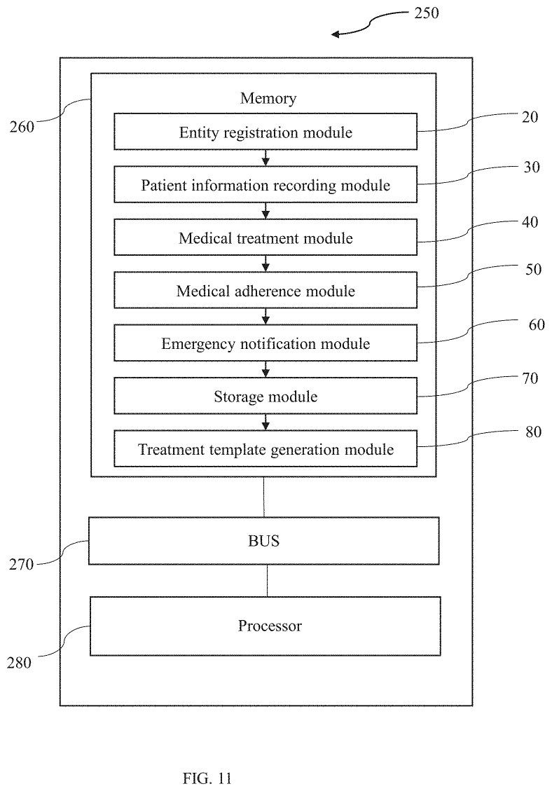

FIG. 11 is a block diagram of a computer or a server 250 in accordance with an embodiment of the present disclosure. The server 250 includes processor(s) 280, and memory 260 coupled to the processor(s) 280.

The processor(s) 280, as used herein, means any type of computational circuit, such as, but not limited to, a microprocessor, a microcontroller, a complex instruction set computing microprocessor, a reduced instruction set computing microprocessor, a very long instruction word microprocessor, an explicitly parallel instruction computing microprocessor, a digital signal processor, or any other type of processing circuit, or a combination thereof.

The memory 260 includes a plurality of modules stored in the form of executable program which instructs the processor 280 via a bus 270 to perform the method steps illustrated in FIG. 1. The memory 260 has following modules: entity registration module 20, the patient information recording module 30, the medical treatment module 40, the medical adherence module 50, the emergency notification module 60, the storage module 70 and the treatment template generation module 80.

The entity registration module 20 is configured to register one or more entities with a plurality of information. The patient information recording module 30 is configured to record information regarding at least one of one or more diseases, one or more bio-physical parameters, type of discomfort, severity of discomfort, part of body experiencing the discomfort and time of discomfort associated with registered patient via at least one of textual input, pre-designated field input and input on 3D image representative of human body. The patient information recording module 30 is also configured to record real-time information regarding the one or more diseases and the one or more bio-physical parameters of the registered patient via IOT enabled one or more wearable sensors.

The medical treatment module 40 is configured to diagnose the one or more diseases based on one or more recorded bio-physical parameters, type of recorded discomfort, severity of recorded discomfort, part of body experiencing the recorded discomfort and time of recorded discomfort associated with the registered patient. The medical treatment module 40 is also configured to generate a real-time treatment plan for the registered patient corresponding to each of one or more diagnosed diseases, based on a pre-designed one or more treatment templates, upon receiving approval from at least one of registered healthcare personnel and the registered doctor. The medical treatment module 40 is also configured to generate a digital prescription for the registered patient according each of the one or more diagnosed diseases and corresponding real-time generated treatment plan.

The medical adherence module 50 is configured to monitor adherence of the registered patient to the real-time generated treatment plan at a micro level and create a medical adherence report. The medical adherence module 50 is also configured to trigger an alert on an event of non-adherence of the real-time generated treatment plan. The medical adherence module 50 is also configured to escalate the alert by changing alert level priority on the event of non-compliance to a triggered alert.

The emergency notification module 60 is configured to notify at least one of the registered patients, the registered healthcare personnel, the registered doctor, registered administrator and registered patient contact about the registered patient adherence to the real-time generated treatment plan. The storage module 70 is configured to store one or more recorded diseases, one or more recorded bio-physical parameters, type of recorded discomfort, severity of recorded discomfort, part of body experiencing the recorded discomfort, time of recorded discomfort associated with registered patient, the real-time generated treatment plan corresponding to the recorded disease, the generated digital prescription corresponding to the recorded disease and created medical adherence report corresponding to the recorded disease.

The medical training module 80 is configured to design one or more treatment templates for a particular disease based on one or more stored diseases, one or more stored bio-physical parameters, type of stored discomfort, severity of stored discomfort, part of body experiencing the stored discomfort, time of stored discomfort associated with registered patient, the real-time generated treatment plan corresponding to the stored disease, generated digital prescription corresponding to the stored disease and created medical adherence report corresponding to the stored disease.

Computer memory elements may include any suitable memory device(s) for storing data and executable program, such as read only memory, random access memory, erasable programmable read only memory, electrically erasable programmable read only memory, hard drive, removable media drive for handling memory cards and the like. Embodiments of the present subject matter may be implemented in conjunction with program modules, including functions, procedures, data structures, and application programs, for performing tasks, or defining abstract data types or low-level hardware contexts. Executable program stored on any of the above-mentioned storage media may be executable by the processor(s) 280.

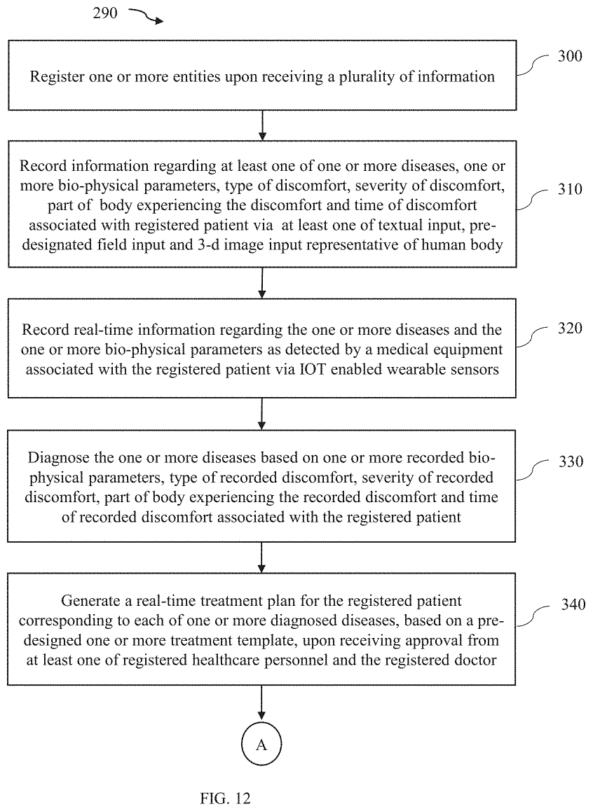

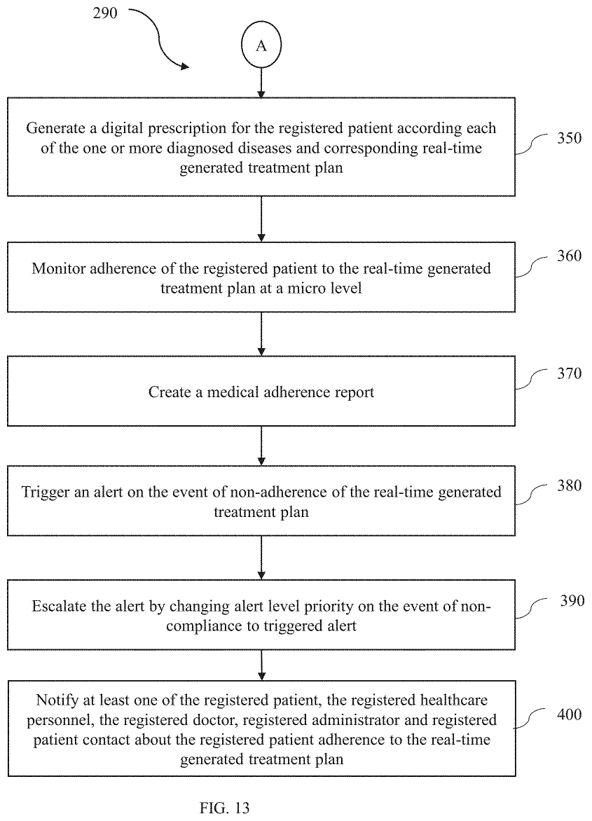

FIG. 12 is a flowchart representing the steps of a method 290 for facilitating medical adherence in accordance with an embodiment of the present disclosure. FIG. 13 is a flowchart representing the steps of the method 290 to facilitate medical adherence in accordance with an embodiment of the present disclosure.

The method 290 also includes registering one or more entities upon receiving a plurality of information in step 300. In one embodiment, registering the one or more entities upon receiving the plurality of information includes registering the one or more entities upon receiving the plurality of information by an entity registration module. In another embodiment, registering the one or more entities upon receiving the plurality of information includes registering the one or more entities comprising comprises a patient, a healthcare personnel, a doctor, an administrator and a patient contact.

The method 290 also includes recording information regarding at least one of one or more diseases, one or more bio-physical parameters, type of discomfort, severity of discomfort, part of body experiencing the discomfort and time of discomfort associated with registered patient via at least one of textual input, pre-designated field input and 3-d image input representative of human body in step 310. In one embodiment, recording the information regarding at least one of the one or more diseases, the one or more bio-physical parameters, the type of discomfort, the severity of discomfort, the part of body experiencing the discomfort and the time of discomfort associated with registered patient via at least one of textual input, pre-designated field input and 3-d image input representative of human body includes recording the information by a patient information recording module.

The method 290 also recording real-time information regarding the one or more diseases and the one or more bio-physical parameters of the registered patient via IOT enabled one or more wearable sensors in step 320. In one embodiment, recording the real-time information regarding the one or more diseases and the one or more bio-physical parameters of the registered patient via IOT enabled one or more wearable sensors includes recording the real-time information by the patient information recording module.

The method 290 also includes diagnosing the one or more diseases based on one or more recorded bio-physical parameters, type of recorded discomfort, severity of recorded discomfort, part of body experiencing the recorded discomfort and time of recorded discomfort associated with the registered patient in step 330. In one embodiment, diagnosing the one or more diseases based on the one or more recorded bio-physical parameters, the type of recorded discomfort, the severity of recorded discomfort, the part of body experiencing the recorded discomfort and the time of recorded discomfort associated with the registered patient includes diagnosing the one or more diseases based on the one or more recorded bio-physical parameters, the type of recorded discomfort, the severity of recorded discomfort, the part of body experiencing the recorded discomfort and the time of recorded discomfort associated with the registered patient by a medical treatment module.

The method 290 also generating a real-time treatment plan for the registered patient corresponding to each of one or more diagnosed diseases, based on a pre-designed one or more treatment template, upon receiving approval from at least one of registered healthcare personnel and the registered doctor in step 340. In one embodiment, generating the real-time treatment plan for the registered patient corresponding to each of one or more diagnosed diseases, based on the pre-designed one or more treatment template, upon receiving the approval from at least one of the registered healthcare personnel and the registered doctor includes generating the real-time treatment plan for the registered patient corresponding to each of the one or more diagnosed diseases, based on the pre-designed one or more treatment template, upon receiving the approval from at least one of the registered healthcare personnel and the registered doctor by the medical treatment module.

The method 290 also includes generating a digital prescription for the registered patient according each of the one or more diagnosed diseases and corresponding real-time generated treatment plan in step 350. In one embodiment, generating the digital prescription for the registered patient according each of the one or more diagnosed diseases and corresponding real-time generated treatment plan includes generating the digital prescription for the registered patient according each of the one or more diagnosed diseases and corresponding real-time generated treatment plan by the medical treatment module.

The method 290 also includes monitoring adherence of the registered patient to the real-time generated treatment plan at a micro level in step 360. In one embodiment, monitoring the adherence of the registered patient to the real-time generated treatment plan at the micro level includes monitoring the adherence of the registered patient to the real-time generated treatment plan at the micro level by a medical adherence module.

In another embodiment, monitoring the adherence of the registered patient to the real-time generated treatment plan at the micro level includes monitoring the adherence of the registered patient to the real-time generated treatment plan at the micro level monitoring daily medication intakes, monitoring follow-up treatment compliance, monitoring medical appointments and monitoring ongoing symptoms and side-effects.

The method also 290 includes creating a medical adherence report in step 370. In one embodiment, creating the medical adherence report includes creating the medical adherence report by the medical adherence module.

The method 290 also includes triggering an alert on the event of non-adherence of the real-time generated treatment plan in step 380. In one embodiment, triggering the alert on the event of non-adherence of the real-time generated treatment plan includes triggering the alert on the event of non-adherence of the real-time generated treatment plan by the medical adherence module. In another embodiment, triggering the alert comprises notifying to the registered patient the alert on the event of non-adherence of the real-time generated treatment plan.

The method 290 also includes escalating the alert by changing alert level priority on the event of non-compliance to triggered alert in step 390. In one embodiment, escalating the alert by changing alert level priority on the event of non-compliance to triggered the alert includes escalating the alert by changing the alert level priority on the event of non-compliance to triggered alert by the medical adherence module.

In such embodiment, escalating the alert by changing alert level priority on the event of non-compliance to triggered alert includes escalating a first priority alert to the registered healthcare personnel on the event of non-compliance to the triggered alert. In another such embodiment, escalating the alert by changing alert level priority on the event of non-compliance to triggered alert includes escalating a second priority alert to the registered doctor on the event of non-compliance to the first priority alert.

The method 290 also includes notifying at least one of the registered patients, the registered healthcare personnel, the registered doctor, registered administrator and registered patient contact about the registered patient adherence to the real-time generated treatment plan in step 400. In one embodiment, notifying at least one of the registered patient, the registered healthcare personnel, the registered doctor, registered administrator and the registered patient contact about the registered patient adherence to the real-time generated treatment plan includes notifying at least one of the registered patient, the registered healthcare personnel, the registered doctor, registered administrator and the registered patient contact about the registered patient adherence to the real-time generated treatment plan by a notification module.

The method 290 also includes storing one or more recorded diseases, one or more recorded bio-physical parameters, type of recorded discomfort, severity of recorded discomfort, part of body experiencing the recorded discomfort, time of recorded discomfort associated with registered patient, the real-time generated treatment plan corresponding to the recorded disease, the generated digital prescription corresponding to the recorded disease and created medical adherence report corresponding to the recorded disease. In one embodiment, storing the one or more recorded diseases, the one or more recorded bio-physical parameters, the type of recorded discomfort, the severity of recorded discomfort, the part of body experiencing the recorded discomfort, the time of recorded discomfort associated with registered patient, the real-time generated treatment plan corresponding to the recorded disease, the generated digital prescription corresponding to the recorded disease and created medical adherence report corresponding to the recorded disease includes storing by a storage module.

The method 290 also includes comprising designing one or more treatment templates for a particular disease based on the one or more stored diseases, one or more analysed bio-physical parameters, type of stored discomfort, severity of stored discomfort, part of body experiencing the stored discomfort, time of stored discomfort associated with registered patient, the real-time generated treatment plan corresponding to the stored disease, generated digital prescription corresponding to the analysed disease and created medical adherence report corresponding to the stored disease. In one embodiment, designing one or more treatment templates for the particular disease based on one or more stored diseases, the one or more stored bio-physical parameters, the type of stored discomfort, the severity of stored discomfort, the part of body experiencing the stored discomfort, the time of stored discomfort associated with registered patient, the real-time generated treatment plan corresponding to the stored disease, the generated digital prescription corresponding to the stored disease and the created medical adherence report corresponding to the stored disease includes designing by the medical treatment module.

The method 290 also includes textual or call interacting facility among the one or more registered entities. In one embodiment, textual or call interacting facility among the one or more registered entities includes textual or call interacting by an entity interaction module.

The method 290 also includes facilitating exchange of real-time feedbacks among the one or more registered entities, wherein the real-time feedbacks enable constant monitoring the registered patient health condition. In one embodiment, facilitating exchange of the real-time feedbacks among the one or more registered entities includes facilitating exchange of the real-time feedbacks among the one or more registered entities by a feedback module.

The method 290 also includes generating a health report based on the real-time generated treatment plan, the generated digital prescription and the created adherence report. In one embodiment, generating the health report based on the real-time generated treatment plan, the generated digital prescription and the created adherence report includes generating the health report based on the real-time generated treatment plan, the generated digital prescription and the created adherence report by health report generation module.

The method 290 also includes storing medical data corresponding to one or more diseases from a plurality of patients in at least one of a canonical and non-canonical data models. In one embodiment, storing the medical data corresponding to one or more diseases from a plurality of patients in at least one of the canonical and the non-canonical data models includes storing by a research repository and sharing module.

The method 290 also includes categorising the stored medical data as per privacy levels. In one embodiment, categorising the stored medical data as per the privacy levels includes categorising the stored medical data as per the privacy levels by the research repository and sharing module.

The method 290 also includes providing access to the stored medical data, based on a clearance with respect to predefined privacy levels, to one or more entities, wherein the privacy levels being defined with respect to privacy concerns and research requirements. In one embodiment, providing access to the stored medical data, based on the clearance with respect to predefined privacy levels, to the one or more entities, includes providing access to the stored medical data, based on the clearance with respect to predefined privacy levels, to the one or more entities by the research repository and sharing module, wherein the privacy levels being defined with respect to privacy concerns and research requirements.

Present disclosure of a system to facilitate medical adherence provides real time medical adherence facility with respect to any patient. Real time medical information of the patient may be easily tracked and monitor by the system. The system registers medical information from the patient as well as respective doctors and healthcare personnel. After such information gathering, the system enables the doctors and healthcare personnel to create real time treatment plan. Digital prescription may also be offered by the system.

The system via alerts and constant monitoring, enforces adherence to created medical treatment plan. The system thereby enables real-time engagement between care-providers and patients throughout the life cycle of the treatment.

The system may also point out missed appointments, missed actions and the like with emergency notifications. As the system maintains and stores all medical information of registered patients, the system may share the data to medical research organizations in-accordance to the need.

While specific language has been used to describe the disclosure, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependant on other acts may be performed in parallel with the other acts. The scope of embodiments is by no means limited by these specific examples.

We claim:

1. A system to facilitate medical adherence, the system comprising:
one or more processors;
an entity registration module operable by the one or more processors, wherein the entity registration module is configured to register one or more entities with a plurality of information, wherein the one or more entities comprises a patient, a healthcare personnel, a doctor, an administrator and a patient contact;
a patient information recording module operable by the one or more processors and operatively coupled to the entry registration module, wherein the patient information recording module is configured to:
record information regarding at least one of one or more diseases, one or more bio-physical parameters, type of discomfort, severity of discomfort, part of body experiencing the discomfort and time of discomfort associated with registered patient via at least one of textual input, pre-designated field input and input on 3D image representative of human body; and
record real-time information regarding the one or more diseases and the one or more bio-physical parameters of the registered patient via IOT enabled one or more wearable sensors;
a medical treatment module operable by the one or more processors, and operatively coupled to the patient information recording module, wherein the medical treatment module is configured to:
diagnose the one or more diseases based on one or more recorded bio-physical parameters, type of recorded discomfort, severity of recorded discomfort, part of body experiencing the recorded discomfort and time of recorded discomfort associated with the registered patient;
generate a real-time treatment plan for the registered patient corresponding to each of one or more diagnosed diseases, based on a pre-designed one or more treatment templates, upon receiving approval from at least one of registered healthcare personnel and the registered doctor; and
generate a digital prescription for the registered patient according each of the one or more diagnosed diseases and corresponding real-time generated treatment plan;
a medical adherence module operable by the one or more processors, and operatively coupled to the medical treatment module, wherein the medical adherence module is configured to:
monitor adherence of the registered patient to the real-time generated treatment plan at a micro level and create a medical adherence report, wherein the adherence comprises,
trigger an alert on an event of non-adherence of the real-time generated treatment plan; and
escalate the alert by changing alert level priority on the event of non-compliance to a triggered alert;
an emergency notification module operable by the one or more processors, and operatively coupled to the medical adherence module, wherein the emergency notification module is configured to notify at least one of the registered patient, the registered healthcare personnel, the registered doctor, registered administrator and registered patient contact about the registered patient adherence to the real-time generated treatment plan;
a storage module operable by the one or more processors and operatively coupled to emergency notification module, wherein the storage module is configured to store one or more recorded diseases, one or more recorded bio-physical parameters, type of recorded discomfort, severity of recorded discomfort, part of body experiencing the recorded discomfort, time of recorded discomfort associated with registered patient, the real-time generated treatment plan corresponding to the recorded disease, the generated digital prescription corresponding to the recorded disease and created medical adherence report corresponding to the recorded disease; and
a treatment template generation module operable by the one or more processors and operatively coupled to the storage module, wherein the medical template generation module is configured to design one or more treatment templates for a particular disease based on one or more analysed diseases, one or more analysed bio-physical parameters, type of analysed discomfort, severity of analysed discomfort, part of body experiencing the analysed discomfort, time of analysed discomfort associated with registered patient, the real-time generated treatment plan corresponding to the analysed disease, generated digital prescription corresponding to the analysed disease and created medical adherence report corresponding to the analysed disease.

2. The system as claimed in claim 1, comprising an entity interaction module operable by the one or more processors and operatively coupled to the medical adherence module, wherein the entity interaction module is configured to provide textual or call interaction facility among the one or more registered entities.

3. The system as claimed in claim 1, comprising a feedback module operable by the one or more processors and operatively coupled to the medical adherence module, wherein the feedback module is configured to facilitate exchange of real-time feedbacks among the one or more registered entities, wherein the real-time feedbacks enable constant monitoring the registered patient health condition.

4. The system as claimed in claim 1, comprising a health report generation module operable by the one or more processors and operatively coupled to the emergency notification module, wherein the health report generation module is configured to generate a health report based on the real-time generated treatment plan, the generated digital prescription and the created adherence report.

5. The system as claimed in claim 1, comprising a research repository and sharing module operable by the one or more processors, and operatively coupled to the medical adherence module, wherein the research repository and sharing module is configured to
store medical data corresponding to one or more diseases from a plurality of patients in at least one of a canonical and non-canonical data models,
categorise the stored medical data as per privacy levels, and
provide access to the stored medical data, based on a clearance with respect to predefined privacy levels, to one or more entities, wherein the privacy levels being defined with respect to privacy concerns and research requirements.

6. The system as claimed in claim 1, wherein the trigger of the alert on the event of non-adherence of the real-time generated treatment plan is notified to the registered patient, wherein on the event of non-compliance to the triggered alert the medical adherence module being escalated to a first priority alert to the registered healthcare personnel, wherein on the event of non-compliance to the first priority alert the medical adherence module being further escalated to a second priority alert to the registered doctor.

7. The system as claimed in claim 1, comprising a triage module configured to categorise the plurality of parties based on degrees of urgency.

8. A method for facilitating medical adherence, the method comprising:
registering, by an entity registration module, one or more entities upon receiving a plurality of information;
recording, by a patient information recording module, information regarding at least one of one or more diseases, one or more bio-physical parameters, type of discomfort, severity of discomfort, part of body experiencing the discomfort and time of discomfort associated with registered patient via at least one of textual input, pre-designated field input and 3-d image input representative of human body;
recording, by the patient information recording module, real-time information regarding the one or more diseases and the one or more bio-physical parameters of the registered patient via IOT enabled one or more wearable sensors;
diagnosing, by a medical treatment module, the one or more diseases based on one or more recorded bio-physical parameters, type of recorded discomfort, severity of recorded discomfort, part of body experiencing the recorded discomfort and time of recorded discomfort associated with the registered patient;
generating, by the medical treatment module, a real-time treatment plan for the registered patient corresponding to each of one or more diagnosed diseases, based on a pre-designed one or more treatment template, upon receiving approval from at least one of registered healthcare personnel and the registered doctor;
generating, by the medical treatment module, a digital prescription for the registered patient according each of the one or more diagnosed diseases and corresponding real-time generated treatment plan;
monitoring, by a medical adherence module, adherence of the registered patient to the real-time generated treatment plan at a micro level;
creating, by the medical adherence module, a medical adherence report;
triggering, by the medical adherence module, an alert on the event of non-adherence of the real-time generated treatment plan;
escalating, by the medical adherence module, the alert by changing alert level priority on the event of non-compliance to triggered alert; and
notifying, by the emergency notification module, at least one of the registered patient, the registered healthcare personnel, the registered doctor, registered administrator and registered patient contact about the registered patient adherence to the real-time generated treatment plan.

9. The method as claimed in claim 8, comprising storing, by a storage module, one or more recorded diseases, one or more recorded bio-physical parameters, type of recorded discomfort, severity of recorded discomfort, part of body experiencing the recorded discomfort, time of recorded discomfort associated with registered patient, the real-time generated treatment plan corresponding to the recorded disease, the generated digital prescription corresponding to the recorded disease and created medical adherence report corresponding to the recorded disease.

10. The method as claimed in claim 8, comprising designing, by the medical training module, one or more treatment templates for a particular disease based on one or more analysed diseases, one or more analysed bio-physical parameters, type of analysed discomfort, severity of analysed discomfort, part of body experiencing the analysed discomfort, time of analysed discomfort associated with registered patient, the real-time generated treatment plan corresponding to the analysed disease, generated digital prescription corresponding to the analysed disease and created medical adherence report corresponding to the analysed disease.

11. The method as claimed in claim 8, comprising textual or call interacting facility, by an entity interaction module, among the one or more registered entities.

12. The method as claimed in claim 8, comprising facilitating, by feedback module, exchange of real-time feedbacks among the one or more registered entities, wherein the real-time feedbacks enable constant monitoring the registered patient health condition.

13. The method as claimed in claim 8, comprising generating, by a health report generation module, a health report based on the real-time generated treatment plan, the generated digital prescription and the created adherence report.

14. The method as claimed in claim 8, comprising storing, by a research repository and sharing module, medical data corresponding to one or more diseases from a plurality of patients in at least one of a canonical and non-canonical data models.

15. The method as claimed in claim 8, comprising categorising, by the research repository and sharing module, the stored medical data as per privacy levels.

16. The method as claimed in claim 8, comprising providing, by the research repository and sharing module, access to the stored medical data, based on a clearance with respect to predefined privacy levels, to one or more entities, wherein the privacy levels being defined with respect to privacy concerns and research requirements.

17. The method as claimed in claim 8, wherein registering, by the entity registration module, the one or more entities comprises a patient, a healthcare personnel, a doctor, an administrator and a patient contact.

18. The method as claimed in claim 8, wherein monitoring, by the medical adherence module, the adherence comprises monitoring daily medication intakes, monitoring follow-up treatment compliance, monitoring medical appointments and monitoring ongoing symptoms and side-effects.

19. The method as claimed in claim 8, wherein triggering, by the medical adherence module, the alert comprises notifying to the registered patient the alert on the event of non-adherence of the real-time generated treatment plan.

20. The method as claimed in claim 8, wherein escalating, by the medical adherence module, the alert comprises escalating a first priority alert to the registered healthcare personnel on the event of non-compliance to the triggered alert.

21. The method as claimed in claim 20, wherein escalating, by the medical adherence module, the alert comprises escalating a second priority alert to the registered doctor on the event of non-compliance to the first priority alert.
